(19) United States
(12) Patent Application Publication
(10) Pub. No.: US 2005/0043629 A1
(43) Pub. Date: Feb. 24, 2005

(54) Apparatus and method for an ultrasonic medical device having a probe with a small proximal end

(75) Inventors: Robert A. Rabiner, North Reading, MA (US); Bradley A. Hare, Chelmsford, MA (US)

Correspondence Address: PALMER & DODGE, LLP;RICHARD B. SMITH, 111 HUNTINGTON AVENUE, BOSTON, MA, 02199, US

(73) Assignee: OmniSonics Medical Technologies, Inc.

(21) Appl. No.: 10/959,703

(22) Filed: Oct. 6, 2004

Related U.S. Application Data
(63) Continuation-in-part of application No. 10/371,781, filed on Feb. 21, 2003.
Application No. 10/371,781 is a continuation of application No. 09/618,352, filed on Jul. 19, 2000, now Pat. No. 6,551,337.
(60) Provisional application No. 60/178,901, filed on Jan. 28, 2000.
(60) Provisional application No. 60/157,824, filed on Oct. 5, 1999.

(51) Int. Cl. A61B 8/14

(52) U.S. Cl. 600/459

(57) ABSTRACT

An apparatus and method for an ultrasonic medical device having an ultrasonic probe with a small proximal end to facilitate over the ultrasonic probe exchanges in a time efficient manner. The ultrasonic probe is inserted into a vasculature and moved to a treatment site of an occlusion. A coupling engaging the ultrasonic probe to a transducer is disengaged to expose a small diameter at the proximal end of the ultrasonic probe. A vascular intervention device is placed over the small diameter at the proximal end and moved along a longitudinal axis of the ultrasonic probe while the ultrasonic probe remains in an approximately fixed position in the vasculature. In a preferred embodiment, the ultrasonic probe acts a guidewire for over the ultrasonic probe exchanges of various vascular intervention devices.

### RELATED APPLICATIONS

This application is a continuation-in-part of application Ser. No. 10/371,781, filed Feb. 21, 2003, which is a continuation of application Ser. No. 09/618,352, filed Jul. 19, 2000, now U.S. Pat. No. 6,551,337, which claims the benefit of Provisional Application Ser. No. 60/178,901, filed Jan. 28, 2000, and claims the benefit of Provisional Application Ser. No. 60/157,824, filed Oct. 5, 1999, the entirety of all these applications are hereby incorporated herein by reference.

### FIELD OF THE INVENTION

The present invention relates to an ultrasonic medical device, and more particularly to an apparatus and method an ultrasonic medical device having a probe with a small proximal end for permitting over the probe transfers of vascular intervention devices.

### BACKGROUND OF THE INVENTION

Vascular occlusive disease affects millions of individuals worldwide and is characterized by a dangerous blockage of blood vessels. Vascular occlusive disease includes thrombosed hemodialysis grafts, peripheral artery disease, deep vein thrombosis, coronary artery disease and stroke. Vascular occlusions (including, but not limited to, clots, intravascular blood clots or thrombus, occlusional deposits, such as calcium deposits, fatty deposits, atherosclerotic plaque, cholesterol buildup, fibrous material buildup and arterial stenoses) result in the restriction or blockage of blood flow in the vessels in which they occur. Occlusions result in oxygen deprivation (“ischemia”) of tissues supplied by these blood vessels. Prolonged ischemia results in permanent damage of tissues which can lead to myocardial infarction, stroke, or death. Targets for occlusion include coronary arteries, peripheral arteries and other blood vessels.

The disruption of an occlusion or thrombus can be affected by pharmacological agents, mechanical means or ultrasonic energy. Many thrombolytic drugs are associated with side effects such as severe bleeding which can result in a cerebral hemorrhage. Mechanical methods of treating thrombolysis include balloon angioplasty and stenting.

Mechanical means of removing an occlusion of biological material include angioplasty and stenting. Angioplasty is also referred to as balloon angioplasty or PTCA—percutaneous transluminal coronary angioplasty. Balloon angioplasty is a minimally invasive, non-surgical way of treating an occlusion of a biological material to remove the biological material and open the vasculature to allow blood to circulate. There are several methods of balloon angioplasty in the prior art. In one method, a catheter is inserted into the vasculature of the body and an x-ray of the vasculature is taken to measure the extent of the narrowing of the vasculature. After the blockage is located, a guidewire is advanced to the site of the occlusion and a second catheter with a balloon located on it is passed over the guidewire. The second catheter is advanced to the occlusion and the balloon is inflated. The inflation of the balloon presses the biological material against the walls of the vasculature and the balloon is subsequently deflated. The inflation and deflation of the balloon may be repeated several times to remove the occlusion of the biological material to increase blood flow.

Stenting is a catheter based procedure in which a stent is inserted into a vasculature of a body. Often, stenting is performed in conjunction with other catheter based procedures including, but not limited to, balloon angioplasty and atherectomy. A stent is a tube made of metal wire or plastic that is inserted into the vasculature of the body to keep the vasculature open and prevent closure of the vasculature. A stent is a permanent device that becomes a part of the cardiovascular system. In one embodiment of a stenting procedure, a guiding catheter is advanced through a sheath to a site of the occlusion of biological material. A stent with a balloon-tipped catheter inside the walls of the stent is advanced to the site of the occlusion of biological material and the balloon is inflated to expand the stent. The expansion of the stent allows the stent to engage to the wall of the vasculature. The balloon catheter is removed while the stent remains engaged to the walls of the vasculature.

The use of ultrasonic probes using ultrasonic energy to fragment body tissue have been used in many surgical procedures (see, e.g., U.S. Pat. No. 5,112,300; U.S. Pat. No. 5,180,363; U.S. Pat. No. 4,989,583; U.S. Pat. No. 4,931,047; U.S. Pat. No. 4,922,902; and U.S. Pat. No. 3,805,787). The use of ultrasonic energy has been proposed both to mechanically disrupt clots, and to enhance the intravascular delivery of drugs to clot formations (see, e.g., U.S. Pat. No. 5,725,494; U.S. Pat. No. 5,728,062; and U.S. Pat. No. 5,735,811). Ultrasonic devices used for vascular treatments typically comprise an extracorporeal transducer coupled to a solid metal wire which is then threaded through the blood vessel and placed in contact with the occlusion (see, e.g., U.S. Pat. No. 5,269,297). In some cases, the transducer, comprising a bendable plate, is delivered to the site of the clot (see, e.g., U.S. Pat. No. 5,931,805).

Some ultrasonic devices include a mechanism for irrigating an area where the ultrasonic treatment is being performed (e.g., a body cavity or lumen) in order to wash tissue debris from the area of treatment. Mechanisms used for irrigation or aspiration described in the art are generally structured such that they increase the overall cross-sectional profile of the elongated probe, by including inner and outer concentric lumens within the probe to provide irrigation and aspiration channels. In addition to making the probe more invasive, prior art probes also maintain a strict orientation of the aspiration and the irrigation mechanism, such that the inner and outer lumens for irrigation and aspiration remain in a fixed position relative to one another, which is generally closely adjacent to the area of treatment. Thus, the irrigation lumen does not extend beyond the suction lumen (i.e., there is no movement of the lumens relative to one another) and any aspiration is limited to picking up fluid and/or tissue remnants within the defined area between the two lumens.

Whether the treatment of the vascular occlusive disease is through mechanical methods, ultrasonic energy methods or through the use of pharmacological agents, the treatment requires the exchange of various vascular intervention devices within the vasculature. Since a surgeon is gaining access to the vasculature and inserting vascular intervention devices into the vasculature, it is important in the treatment of the vascular occlusive disease that the treatment time be minimized. However, navigating a vascular intervention device to a site of an occlusion can be both a challenging and time consuming process for a surgeon. The outside diameters of many medical devices are large, thereby making it difficult to move the medical device to a treatment site without a guiding mechanism to assist the surgeon.

In many surgical procedures, a probe is delivered to a site of the occlusion in a vasculature and a vascular intervention device is moved over the probe while the probe is in an approximately fixed position inside the vasculature of a body. Delivering a vascular intervention device over the probe requires the diameter at the proximal end of the probe be small so the vascular intervention device can be moved over the proximal end of the probe and moved along a longitudinal axis of the probe. In order to amplify the ultrasonic energy, many probes have a horn assembly at the proximal end. Ultrasonic energy is transmitted to the horn assembly by a source or generator that is engaged to the horn assembly at the proximal end of the horn assembly. The horn assemblies are large in size and do not allow a vascular intervention device to be placed over the horn assembly.

U.S. Pat. No. 5,269,297 to Weng et al. discloses an ultrasonic transmission apparatus to transmit ultrasonic energy from a source to a distal tip with minimal energy loss. The Weng et al. device includes a horn connected to an energy source for amplifying ultrasound displacement and a transmitter for transmitting ultrasonic energy at a frequency. The Weng et al. device comprises a proximal end with a large diameter and a plurality of diameter transitions that would not allow a vascular intervention device to be placed over the proximal end of the Weng et al. device. In addition, the Weng et al. device does not have a quick attachment-detachment system that would allow for a medical device to be placed over the Weng et al. device.

U.S. Pat. No. 5,971,949 to Levin et al. discloses an ultrasound transmission apparatus and method of using the same to treat intravascular conditions with an ultrasonic probe having a proximal end, a distal end and an ultrasonic energy source. The Levin et. al device has a proximal end with a large diameter of approximately 0.5 inches and the Levin et al. device does not have a quick attachment and detachment system whereby the probe can remain in an approximately fixed position within a vasculature of a body, while the ultrasonic source is removed and a medical device is placed over the probe at a location with a smaller diameter.

The prior art devices and methods of delivering a vascular intervention device over an ultrasonic probe to a location adjacent to a site of an occlusion are inadequate and time consuming. Some prior art probes have a large diameter at the proximal end that a vascular intervention device could not fit over. Prior art probes have a large diameter at the proximal end that would require the prior art probe be removed from the vasculature before delivering the vascular intervention device over the probe and to the site of the occlusion. Prior art probes do not have a quick attachment and detachment system that allows a surgeon to remove the ultrasonic energy source in order for the surgeon to move a vascular intervention device along a longitudinal axis of an ultrasonic probe to the site of the occlusion while the ultrasonic probe remains in a fixed position in the vasculature.

### SUMMARY OF THE INVENTION

The present invention provides an apparatus and a method for an ultrasonic medical device having a probe with a small proximal end. The present invention is an ultrasonic medical device comprising an ultrasonic probe having a proximal end, a distal end and a longitudinal axis therebetween. The ultrasonic medical device includes a transducer having a proximal end and a distal end, the transducer transmitting an ultrasonic energy to the ultrasonic probe. The ultrasonic medical device also includes a coupling that engages the proximal end of the ultrasonic probe to the distal end of the transducer and an ultrasonic energy source engaged to the transducer that produces an ultrasonic energy. The proximal end of the ultrasonic probe has a small diameter that allows a vascular intervention device to be placed over the proximal end of the ultrasonic probe and moved along the longitudinal axis of the ultrasonic probe while the ultrasonic probe remains with a vasculature of a body.

The present invention is an elongated ultrasonic probe comprising a proximal end, a distal end terminating in a probe tip and a longitudinal axis therebetween. The elongated ultrasonic probe includes a small diameter at the proximal end that allows a first vascular intervention device to be placed over the proximal end of the elongated ultrasonic probe and moved along the longitudinal axis of the elongated ultrasonic probe while the distal end of the elongated ultrasonic probe remains in a vasculature of a body.

The present invention is a method of placing a first vascular intervention device over an ultrasonic probe and moving the first vascular intervention device to a treatment site to ablate an occlusion. The ultrasonic probe is inserted into a vasculature of a body and moved to the treatment site. A coupling that engages a proximal end of the ultrasonic probe to a transducer is disengaged to expose a proximal end of the ultrasonic probe. The first vascular intervention device is placed over a small diameter at the proximal end of the ultrasonic probe and the first vascular intervention device is moved along a longitudinal axis of the ultrasonic probe so the first vascular intervention device is adjacent to the treatment site while the ultrasonic probe remains in an approximately fixed position in the vasculature. The coupling is re-engaged to engage the proximal end of the ultrasonic probe to the transducer and an ultrasonic energy source engaged to the ultrasonic probe is activated to produce an ultrasonic energy to ablate the occlusion.

The present invention is a method of exchanging vascular intervention devices within a vasculature of a body comprising: inserting a first vascular intervention device into the vasculature; delivering a flexible ultrasonic probe inside of the first vascular intervention device to a treatment site; moving a second vascular intervention device over a proximal end of the flexible ultrasonic probe while the flexible ultrasonic probe remains in an approximately fixed position in the vasculature; and moving the second vascular intervention device within an interior of the first vascular intervention device along a longitudinal axis of the flexible ultrasonic probe to the treatment site.

The present invention provides an apparatus and a method for an ultrasonic medical device having a probe with a small proximal end to facilitate an over the probe exchange of one or more vascular intervention devices. The ultrasonic probe is inserted into a vasculature, moved to a treatment site and the proximal end of the ultrasonic probe is exposed. A vascular intervention device is moved over the proximal end of the ultrasonic probe and moved along a longitudinal axis of the ultrasonic probe to the treatment site. The present invention provides an ultrasonic medical device that is simple, user-friendly, time efficient, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a view of an ultrasonic medical device of the present invention with an ultrasonic probe inserted into a vasculature in an arm of a patient.

FIG. 2A is a side plan view of an ultrasonic probe of the present invention capable of operating in a transverse mode.

FIG. 2B is a side plan view of an ultrasonic probe of the present invention having an approximately uniform diameter from a proximal end of the ultrasonic probe to the distal end of the ultrasonic probe.

FIG. 3 is a view of an ultrasonic probe of the present invention with a quick attachment-detachment system and a portion of a transducer.

FIG. 4 is a side plan view of an ultrasonic probe of the present invention with a first vascular intervention device placed over a distal end of the ultrasonic probe.

FIG. 5 is a side plan view of an ultrasonic medical device of the present invention with a first vascular intervention device placed over an ultrasonic probe.

FIG. 6 is a side plan view of an ultrasonic medical device of the present invention showing a plurality of transverse nodes and a plurality of transverse anti-nodes along a portion of a longitudinal axis of an ultrasonic probe.

FIG. 7 is a fragmentary side plan view of an ultrasonic probe of the present invention with a first vascular intervention device located at a distal end of the ultrasonic probe and a second vascular intervention device comprising a stent located at a proximal end of the ultrasonic probe.

FIG. 8 is a view of an ultrasonic medical device of the present invention with a first vascular intervention device and a second vascular intervention device comprising a stent located over an ultrasonic probe.

FIG. 9 is a view of an ultrasonic medical device of the present invention with a first vascular intervention device and an alternative second vascular intervention device located over an ultrasonic probe.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for an ultrasonic medical device having a probe with a small proximal end. An ultrasonic medical device comprises the ultrasonic probe with a proximal end having a small diameter that allows a vascular intervention device to be placed over the proximal end and moved along a longitudinal axis of the ultrasonic probe without removing the ultrasonic probe from within a vasculature of a body. The ultrasonic medical device includes a coupling that engages the proximal end of the ultrasonic probe to a distal end of a transducer, allowing the proximal end of the ultrasonic probe to be exposed. The ultrasonic medical device is used to ablate an occlusion in the vasculature. The present invention also provides a method of exchanging a plurality of vascular intervention devices within the vasculature.

The small proximal end of the probe permits the placing of a vascular intervention device over the small proximal end by disengaging an ultrasonic probe from the medical device to expose a small proximal end of the ultrasonic probe of the medical device, moving an vascular intervention device over the small proximal end and along a longitudinal length of the ultrasonic probe to the occlusion, and re-engaging the proximal end of the ultrasonic probe to the medical device.

The following terms and definitions are used herein:

“Ablate” as used herein refers to removing, clearing, destroying or taking away a biological material. “Ablation” as used herein refers to a removal, clearance, destruction, or taking away of the biological material.

“Node” as used herein refers to a region of a minimum energy emitted by an ultrasonic probe at or adjacent to a specific location along a longitudinal axis of the ultrasonic probe.

“Anti-node” as used herein refers to a region of a maximum energy emitted by an ultrasonic probe at or adjacent to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe).

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Vasculature” as used herein refers to the entire circulatory system for the blood supply including the venous system, the arterial system and the associated vessels, arteries, veins, capillaries, blood, and the heart. The arterial system is the means by which blood with oxygen and nutrients is transported to tissues. The venous system is the means by which blood with carbon dioxide and metabolic by-products is transported for excretion.

“Biological material” as used herein refers to a collection of a matter including, but not limited to, a group of similar cells, intravascular blood clots, thrombus, fibrin, occlusions, calcified plaque, calcium deposits, occlusional deposits, atherosclerotic plaque, fatty deposits, adipose tissues, atherosclerotic cholesterol buildup, plaque, fibrous material buildup, arterial stenoses, minerals, high water content tissues, platelets, cellular debris, wastes and other occlusive materials.

“Vascular intervention device” as used herein refers to any medical device which can be inserted into a body including, but not limited to, a catheter, balloon catheter, inflation mechanism, a PTCA balloon, a stent, a stent delivery system, a graft, a stent graft, a drug eluding stent, vascular introducer, lumen, probe, and other similar devices known in the art.

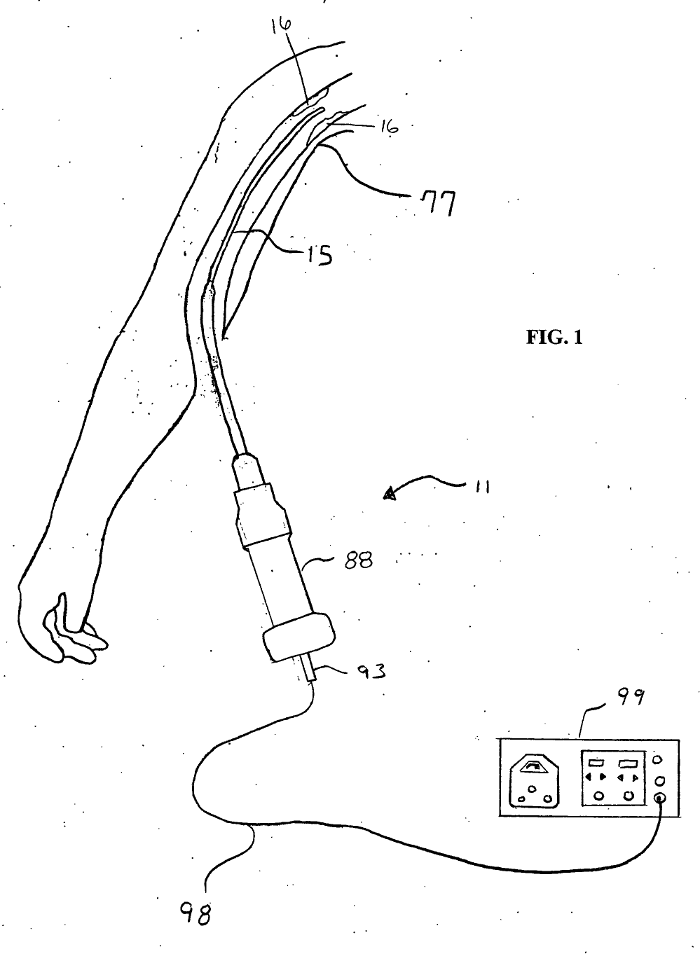

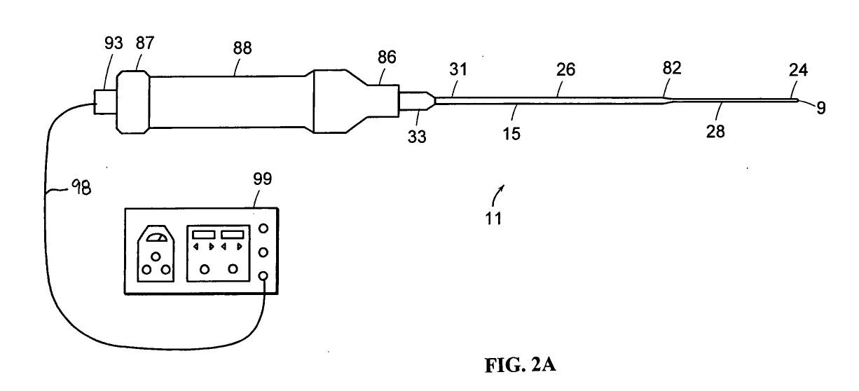

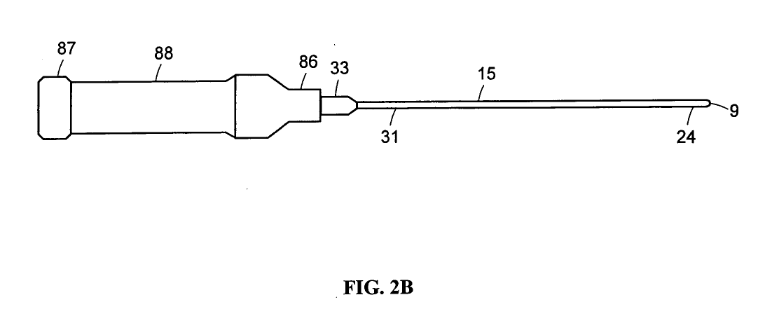

An apparatus for an ultrasonic medical device having a probe with a small proximal end in a general use environment is illustrated generally at 11 in FIG. 1. A more detailed description of the ultrasonic probe 15 is illustrated in FIG. 2A and FIG. 2B. A portion of a longitudinal axis of the ultrasonic probe 15 is inserted into a vasculature of an arm 77. The ultrasonic medical device 11 includes the ultrasonic probe 15 which is coupled to an ultrasonic energy source or generator 99 for the production of an ultrasonic energy. A handle 88, comprising a proximal end 87 and a distal end 86, surrounds a transducer within the handle 88. A connector 93 and a connecting wire 98 engage the ultrasonic energy source 99 to the transducer. The ultrasonic probe 15 includes the proximal end 31 and a distal end 24 that ends in a probe tip 9. In a preferred embodiment of the present invention shown in FIG. 2A, a diameter of the ultrasonic probe 15 decreases from a first defined interval 26 to a second defined interval 28 along a longitudinal axis of the ultrasonic probe 15 over an at least one transition 82. A coupling 33 that engages a proximal end 31 of the ultrasonic probe 15 to the transducer within the handle 88 is illustrated generally in FIGS. 1-3, 5-6, 8-9. In a preferred embodiment of the present invention, the coupling 33 is a quick attachment-detachment system. An ultrasonic medical device with a quick attachment-detachment system is described in the Assignee's U.S. Pat. No. 6,695,782 and Assignee's co-pending patent applications U.S. Ser. No. 10/268,487 and U.S. Ser. No. 10/268,843, and the entirety of all these patents and patent applications are hereby incorporated herein by reference.

FIG. 2B shows an alternative embodiment of the ultrasonic probe 15 of the present invention. In the embodiment of the present invention shown in FIG. 2B, the diameter of the ultrasonic probe 15 is approximately uniform from the proximal end 31 of the ultrasonic probe 15 to the distal end 24 of the ultrasonic probe 15.

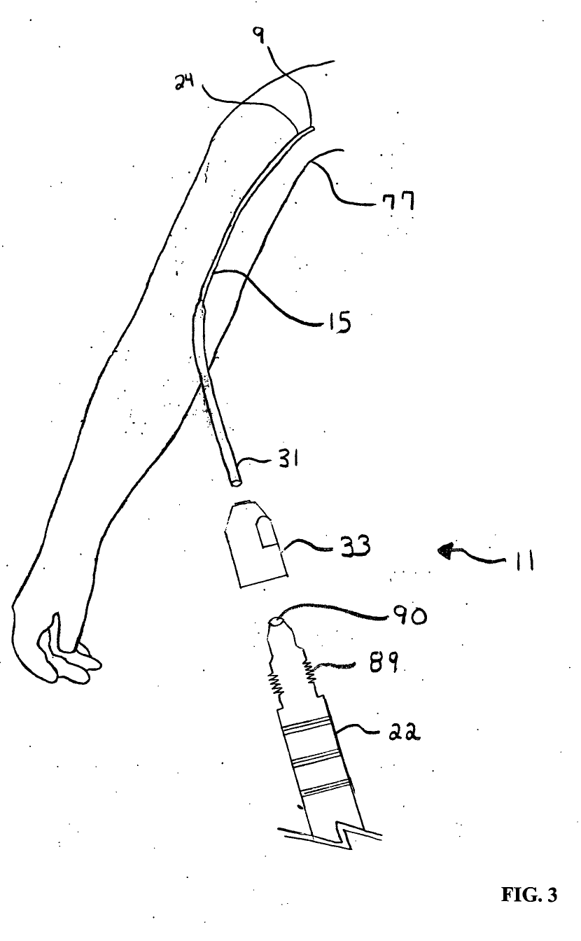

FIG. 3 shows the ultrasonic medical device 11 of the present invention with the ultrasonic probe 15, the coupling 33 and a transducer 22 separated from one another. The transducer 22 has a proximal end, a distal end and a transducer fastener 89. FIG. 3 illustrates the components that are disassembled when exposing the proximal end 31 of the ultrasonic probe 15 and assembled for the functional ultrasonic medical device 11.

A medical professional gains access to the vasculature through an insertion point in the vasculature. A device, including, but not limited to, a vascular introducer can be used to create an insertion point in the vasculature to gain access to the vasculature. A vascular introducer for use with an ultrasonic probe is described in Assignee's co-pending patent application U.S. Ser. No. 10/080,787, and the entirety of this application is hereby incorporated herein by reference.

In a preferred embodiment of the present invention shown in FIG. 1, the ultrasonic probe 15 is inserted into the vasculature in the arm 77 by grasping the handle and inserting the ultrasonic probe 15 into the vasculature and moving the ultrasonic probe 15 to a site of an occlusion (not shown). With the ultrasonic probe 15 at the site of the occlusion, the transducer 22 is disengaged from the proximal end 31 of the ultrasonic probe 15. The coupling 33 disengages the transducer 22 at the transducer fastener 89 by a complementary quick attachment-detachment fastener (not shown) on an inside surface of the coupling 33. The proximal end 31 of the ultrasonic probe is removed from within the transducer tip 90 and the small proximal end 31 of the ultrasonic probe 15 is exposed.

In a preferred embodiment of the present invention, the transducer fastener 89 and the complementary quick attachment-detachment fastener comprise a plurality of threads. Other transducer fasteners and quick attachment-detachment fasteners that could be used for engaging the ultrasonic probe 15 to the transducer 22 include, but are not limited to, adhesives, glues, rivets, blind fasteners, mechanical snaps and other mechanical fasteners. Those skilled in the art will recognize that other methods of engaging the ultrasonic probe to the transducer 22 are known in the art and are within the spirit and scope of the present invention.

By disengaging the ultrasonic probe 15 from the transducer 22, the proximal end 31 of the ultrasonic probe 15 is exposed while the ultrasonic probe 15 remains in the vasculature at the site of the occlusion. The small diameter at the proximal end 31 of the ultrasonic probe 15 allows for at least one vascular intervention device to be placed over the proximal end 31 without removing the ultrasonic probe 15 from the vasculature. Re-engagement of the ultrasonic probe 15 to the transducer 22 with the coupling 33 is done in a time efficient manner. Prior art probes comprise proximal ends with a large diameter that prevent vascular intervention devices from being placed over the ultrasonic probe without removing the ultrasonic probe 15 from the vasculature. By having a proximal end with a large diameter, the treatment time for an occlusion ablation process is longer, the effectiveness of the occlusion ablation is compromised and a patient is subjected to additional health risks.

In an embodiment of the present invention, the diameter of the proximal end 31 of the ultrasonic probe is about 0.012 inches. In another embodiment of the present invention, the diameter of the proximal end 31 of the ultrasonic probe is about 0.025 inches. In other embodiments of the present invention, the diameter of the proximal end 31 of the ultrasonic probe 15 varies between 0.003 inches and about 0.025 inches. In a preferred embodiment of the present invention, the small diameter at the proximal end 31 of the ultrasonic probe 15 is approximately uniform along a length of the proximal end 31 of the ultrasonic probe 15. Those skilled in the art will recognize the ultrasonic probe can have a diameter at the proximal end 31 smaller than about 0.003 inches, larger than about 0.025 inches and between about 0.003 inches and 0.025 inches and be within the spirit and scope of the present invention.

In a preferred embodiment of the present invention, the ultrasonic probe 15 is a wire. In an embodiment of the present invention, the ultrasonic probe 15 is elongated. In a preferred embodiment of the present invention, the diameter of the ultrasonic probe 15 decreases from the first defined interval 26 to the second defined interval 28. In a preferred embodiment of the present invention, the ultrasonic probe 15 has a small diameter. In another embodiment of the present invention, the diameter of the ultrasonic probe 15 decreases at greater than two defined intervals. In a preferred embodiment of the present invention, the transitions 82 of the ultrasonic probe 15 are tapered to gradually change the diameter from the proximal end 31 to the distal end 24 along the longitudinal axis of the ultrasonic probe 15. In another embodiment of the present invention, the transitions 82 of the ultrasonic probe 15 are stepwise to change the diameter from the proximal end 31 to the distal end 24 along the longitudinal axis of the ultrasonic probe 15. Those skilled in the art will recognize that there can be any number of defined intervals and transitions, and that the transitions can be of any shape known in the art and be within the spirit and scope of the present invention.

In a preferred embodiment of the present invention, the diameter of the ultrasonic probe 15 gradually decreases from the proximal end 31 to the distal end 24. In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the gradual change of the diameter from the proximal end 31 to the distal end 24 occurs over at least one transition 82 with each transition 82 having an approximately equal length. In another embodiment of the present invention, the gradual change of the diameter from the proximal end 31 to the distal end 24 occurs over a plurality of transitions 82 with each transition 82 having a varying length. The transition 82 refers to a section where the diameter varies from a first diameter to a second diameter.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. In an embodiment of the present invention, the ultrasonic probe 15 is between about 30 centimeters and about 300 centimeters in length. In an embodiment of the present invention, the ultrasonic probe 15 is a wire. Those skilled in the art will recognize an ultrasonic probe can have a length shorter than about 30 centimeters and a length longer than about 300 centimeters and be within the spirit and scope of the present invention.

The ultrasonic probe has a stiffness that gives the ultrasonic probe 15 a flexibility so it can be bent, flexed and articulated in a vasculature of a body. In the embodiment of the present invention shown in FIGS. 1, 3-5, 7-9, the ultrasonic probe 15 is inserted into a vasculature in the arm 77. In another embodiment of the present invention, the ultrasonic probe 15 is inserted into a leg of the patient. In another embodiment of the present invention, the ultrasonic probe 15 is inserted into a groin of the patient. The ultrasonic probe 15 can be bent, flexed and deflected to reach an occlusion that would otherwise be difficult to reach. Those skilled in the art will recognize the ultrasonic probe can be inserted at several locations of the body and be within the spirit and scope of the present invention.

The probe tip 9 can be any shape including, but not limited to, rounded, bent, a ball or larger shapes. In a preferred embodiment of the present invention, the probe tip 9 is smooth to prevent damage to the arteries and veins of the vasculature. In one embodiment of the present invention, the ultrasonic energy source 99 is a physical part of the ultrasonic medical device 11. In another embodiment of the present invention, the ultrasonic energy source 99 is not an integral part of the ultrasonic medical device 11.

In a preferred embodiment of the present invention, the ultrasonic probe 15 has a small diameter. In a preferred embodiment of the present invention, the cross section of the ultrasonic probe 15 is approximately circular. In another embodiment, the cross section of at least a portion of the ultrasonic probe 15 is non-circular. The ultrasonic probe 15 comprising a wire having a non-circular cross section at the distal end can navigate through the vasculature. The ultrasonic probe 15 comprising a flat wire is steerable in the vasculature. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The ultrasonic probe 15 is inserted into the vasculature and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The ultrasonic probe 15 is designed, constructed and comprised of a material to not dampen the transverse ultrasonic vibration, and thereby supports a transverse vibration when flexed. In a preferred embodiment of the present invention, the ultrasonic probe 15 comprises titanium or a titanium alloy. Titanium is a strong, flexible, low density, low radiopacity and easily fabricated metal that is used as a structural material. Titanium and its alloys have excellent corrosion resistance in many environments and have good elevated temperature properties. In a preferred embodiment of the present invention, the ultrasonic probe 15 comprises titanium alloy Ti-6Al-4V. The elements comprising Ti-6Al-4V and the representative elemental weight percentages of Ti-6Al-4V are titanium (about 90%), aluminum (about 6%), vanadium (about 4%), iron (maximum about 0.25%) and oxygen (maximum about 0.2%). In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel.

In another embodiment of the present invention, the ultrasonic probe 15 comprises a super-elastic alloy. Even when bent or stretched, the super-elastic alloy returns to its original shape when the stress is removed. The ultrasonic probe 15 may comprise super-elastic alloys known in the art including, but not limited to, nickel-titanium super-elastic alloys and Nitinol. Nitinol is a family of intermetallic materials, which contain a nearly equal mixture of nickel and titanium. Other elements can be added to adjust or tune the material properties. Nitinol is less stiff than titanium and is maneuverable in the vasculature. Nitonol has shape memory and super-elastic characteristics. The shape memory effect describes the process of restoring the original shape of a plastically deformed sample by heating it. This is a result of a crystalline phase change known as thermoelastic martensitic transformation. Below the transformation temperature, Nitinol is martensitic. Nitinol's excellent corrosion resistance, biocompatibility, and unique mechanical properties make it well suited for medical devices. Those skilled in the art will recognize that the ultrasonic probe can be comprised of many other materials known in the art and be within the spirit and scope of the present invention.

The handle 88 surrounds the transducer 22 located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer converts electrical energy provided by the ultrasonic energy source 99 to mechanical energy and sets the operating frequency of the ultrasonic medical device 11. The transducer 22 transmits ultrasonic energy received from the ultrasonic energy source 99 to the ultrasonic probe 15. Energy from the ultrasonic energy source 99 is transmitted along the longitudinal axis of the ultrasonic probe 15, causing the ultrasonic probe 15 to vibrate in a transverse mode. The transducer 22 is capable of engaging the ultrasonic probe 15 at the proximal end 31 with sufficient restraint to form an acoustical mass that can propagate the ultrasonic energy provided by the ultrasonic energy source 99.

The ultrasonic energy source 99 produces a transverse ultrasonic vibration along a portion of the longitudinal axis of the ultrasonic probe 15. The ultrasonic probe 15 can support the transverse ultrasonic vibration along the portion of the longitudinal axis of the ultrasonic probe 15. The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction. As a consequence of the transverse ultrasonic vibration of the ultrasonic probe 15, the occlusion destroying effects of the ultrasonic medical device 11 are not limited to those regions of the ultrasonic probe 15 that may come into contact with the occlusion 16. Rather, as a section of the longitudinal axis of the ultrasonic probe 15 is positioned in proximity to an occlusion, a diseased area or lesion, the occlusion 16 is removed in all areas adjacent to a plurality of energetic transverse nodes and transverse anti-nodes that are produced along a portion of the longitudinal axis of the ultrasonic probe 15, typically in a region having a radius of up to about 6 mm around the ultrasonic probe 15.

The transverse ultrasonic vibration of the ultrasonic probe 15 results in a portion of the longitudinal axis of the ultrasonic probe 15 vibrated in a direction not parallel to the longitudinal axis of the ultrasonic probe 15. The transverse vibration results in movement of the longitudinal axis of the ultrasonic probe 15 in a direction approximately perpendicular to the longitudinal axis of the ultrasonic probe 15. Transversely vibrating ultrasonic probes for biological material ablation are described in the Assignee's U.S. Pat. No. 6,551,337; U.S. Pat. No. 6,652,547; U.S. Pat. No. 6,660,013; and U.S. Pat. No. 6,695,781 which further describe the design parameters for such an ultrasonic probe and its use in ultrasonic devices for ablation, and the entirety of these patents and patent applications are hereby incorporated herein by reference.

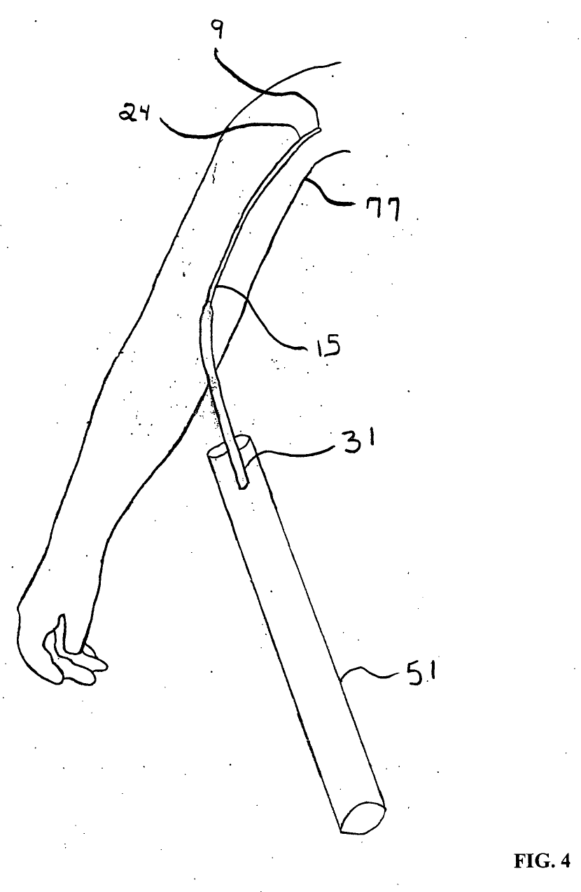

FIG. 4 shows a first vascular intervention device 51 being placed over the proximal end 31 of the ultrasonic probe 15. The small proximal end 31 of the ultrasonic probe 15 allows for the first vascular intervention device 51 to be placed over the proximal end 31 without removing the ultrasonic probe 15 from the vasculature. The ultrasonic probe 15 is a guide for the first vascular intervention device 51. In an embodiment of the present invention, the ultrasonic probe 15 serves as a guidewire.

The ultrasonic probe 15 of the present invention allows the ultrasonic probe 15 to be used as a rail for various vascular intervention devices. The coupling provides a simple and quick way to disengage the ultrasonic probe from the transducer, allowing the vascular intervention device to be slid over the ultrasonic probe. More importantly, the small diameter at the proximal end of the ultrasonic probe allows for a plurality of standard vascular intervention devices to be placed over the proximal end of the ultrasonic probe and slid along the longitudinal axis of the ultrasonic probe to a treatment site. In a preferred embodiment of the present invention, the ultrasonic probe is a guidewire for use as a rail for various vascular intervention devices as well as an occlusion ablation device. In another embodiment of the present invention, the ultrasonic probe of the present invention serves only as a rail for vascular intervention devices.

In an embodiment of the present invention, the first vascular intervention device 51 is a catheter. In another embodiment of the present invention, the first vascular intervention device 51 is a balloon catheter. In other embodiments of the present invention, the first vascular intervention device 51 is selected from a group including, but not limited to, a PTCA balloon, a stent, a stent delivery system, a graft, a stent graft, a drug eluding stent and similar devices. Those skilled in the art will recognize there are several first vascular intervention devices known in the art that are within the spirit and scope of the present invention.

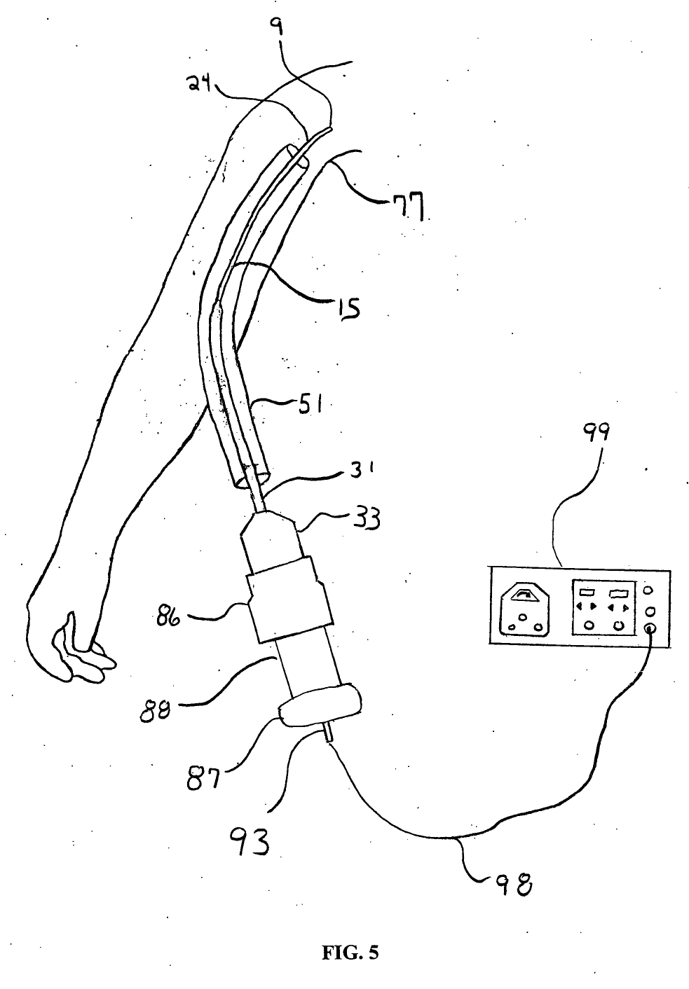

FIG. 5 shows the first vascular intervention device 51 placed over a portion of the longitudinal axis of the ultrasonic probe 15 and located proximal to the site of the occlusion. FIG. 5 shows the ultrasonic medical device 11 in an assembled state where the ultrasonic probe 15 engages the transducer 22 within the handle 88.

With the ultrasonic probe 15 and the first vascular intervention device 51 at the site of the occlusion, the ultrasonic energy source 99 is activated to energize the ultrasonic probe 15. The ultrasonic energy source 99 provides a low power electric signal between about 2 watts to about 15 watts to the transducer 22 that is located within the handle 88. The transducer 22 converts electrical energy provided by the ultrasonic energy source 99 to mechanical energy. The operating frequency of the ultrasonic medical device 11 is set by the transducer and the ultrasonic energy source 99 finds the resonant frequency of the transducer through a Phase Lock Loop. By an appropriately oriented and driven cylindrical array of piezoelectric crystals of the transducer, the horn creates a longitudinal wave along at least a portion of the longitudinal axis of the ultrasonic probe 15. The longitudinal wave is converted to a transverse wave along at least a portion of the longitudinal axis of the ultrasonic probe 15 through a nonlinear dynamic buckling of the ultrasonic probe 15.

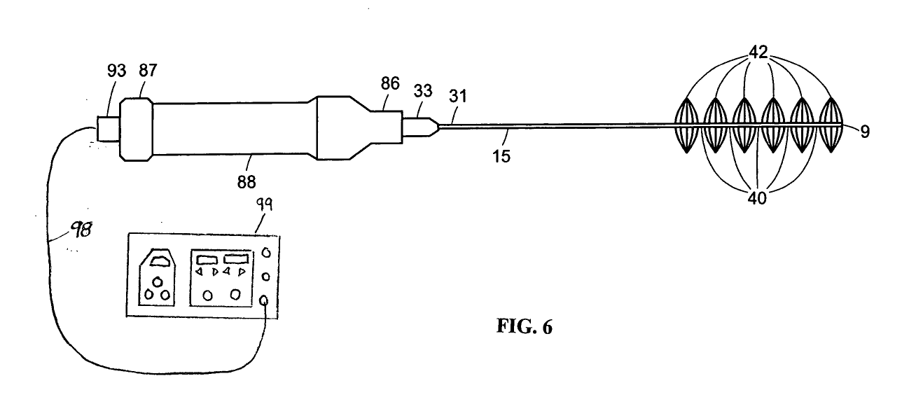

FIG. 6 shows a side plan view of the ultrasonic medical device 11 of the present invention showing a plurality of transverse nodes 40 and a plurality of transverse anti-nodes 42 along a portion of the longitudinal axis of the ultrasonic probe 15. The transverse nodes 40 are areas of minimum energy and minimum vibration. A plurality of transverse anti-nodes 42, or areas of maximum energy and maximum vibration, also occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and transverse anti-nodes 42 is a function of the frequency, and can be affected by tuning the ultrasonic probe 15. In a properly tuned ultrasonic probe 15, the transverse anti-nodes 42 will be found at a position one-half of the distance between the transverse nodes 40 located adjacent to each side of the transverse anti-nodes 42.

The transverse wave is transmitted along the longitudinal axis of the ultrasonic probe 15 and the interaction of the surface of the ultrasonic probe 15 with the medium surrounding the ultrasonic probe 15 creates an acoustic wave in the surrounding medium. As the transverse wave is transmitted along the longitudinal axis of the ultrasonic probe 15, the ultrasonic probe 15 vibrates transversely. The transverse motion of the ultrasonic probe 15 produces cavitation in the medium surrounding the ultrasonic probe 15 to ablate the occlusion 16. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the occlusion 16, while having no damaging effects on healthy tissue.

The occlusion 16 is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

As a consequence of the transverse ultrasonic vibration of the ultrasonic probe 15, the occlusion destroying effects of the ultrasonic medical device 11 are not limited to those regions of the ultrasonic probe 15 that may come into contact with the occlusion 16. Rather, as a section of the longitudinal axis of the ultrasonic probe 15 is positioned in proximity to the occlusion 16, the occlusion 16 is removed in all areas adjacent to the plurality of energetic transverse nodes 40 and transverse anti-nodes 42 that are produced along the portion of the length of the longitudinal axis of the ultrasonic probe 15, typically in a region having a radius of up to about 6 mm around the ultrasonic probe 15. The extent of the acoustic energy produced from the ultrasonic probe 15 is such that the acoustic energy extends radially outward from the longitudinal axis of the ultrasonic probe 15 at the transverse anti-nodes 42 along the portion of the longitudinal axis of the ultrasonic probe 15. In this way, actual treatment time using the transverse mode ultrasonic medical device 11 according to the present invention is greatly reduced as compared to methods disclosed in the prior art that primarily utilize longitudinal vibration (along the axis of the probe).

A novel feature of the present invention is the ability to utilize ultrasonic probes 15 of extremely small diameter compared to prior art probes, without loss of efficiency, because the occlusion fragmentation process is not dependent on the area of the probe tip 9. Highly flexible ultrasonic probes 15 can therefore be designed for facile insertion into occlusion areas or narrow interstices that contain the occlusion 16. Another advantage provided by the present invention is the ability to rapidly move the occlusion 16 from large areas within cylindrical or tubular surfaces.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of occlusion destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the occlusion selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 10 kHz to about 100 kHz. In a preferred embodiment of the present invention, the frequency of ultrasonic energy is from about 20 kHz to about 40 kHz. Frequencies in this range are specifically destructive of occlusions including, but not limited to, hydrated (water-laden) tissues such as endothelial tissues, while substantially ineffective toward high-collagen connective tissue, or other fibrous tissues including, but not limited to, vascular tissues, epidermal, or muscle tissues.

The present invention allows for a plurality of vascular intervention devices to be used in a treatment procedure. In an embodiment of the present invention, the plurality of vascular intervention devices are exchanged within the vasculature of the body. The ultrasonic probe 15 is a guide for the plurality of vascular intervention devices.

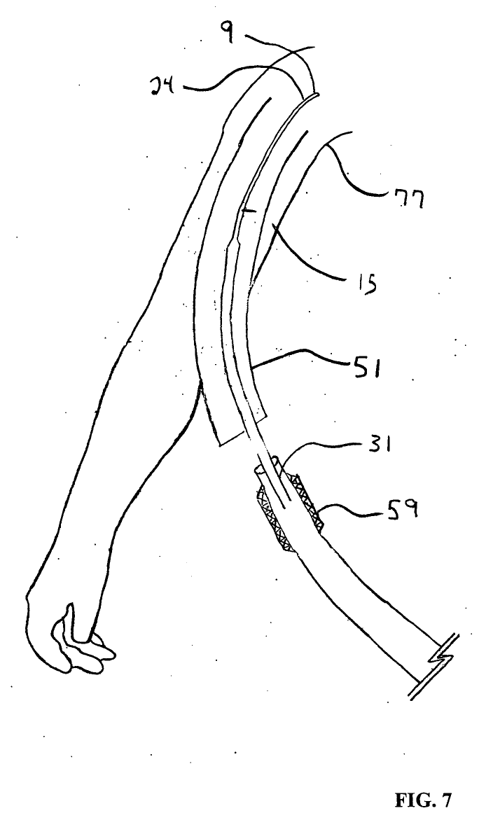

FIG. 7 shows an ultrasonic probe 15 in the vasculature of the arm 77 with a first vascular intervention device 51 located over a portion of the longitudinal axis of the ultrasonic probe 15 and a second vascular intervention device 59 placed over the proximal end 31 of the ultrasonic probe 15. FIG. 7 illustrates a step of exchanging a plurality of vascular intervention devices in the vasculature. The small diameter at the proximal end 31 of the ultrasonic probe 15 allows for the second vascular intervention device 59 to be placed over the proximal end 31 of the ultrasonic probe 15. The second vascular intervention device 59 is moved within the first vascular intervention device 51.

In the embodiment of the present invention shown in FIG. 7, the second vascular intervention device 59 comprises a stent. In another embodiment of the present invention, the second vascular intervention device is a balloon catheter. In another embodiment of the present invention, the second vascular intervention device 59 is a catheter. In other embodiments of the present invention, the second vascular intervention device 59 is selected from a group including, but not limited to, a PTCA balloon, a drug eluding stent, a probe, a lumen and similar devices. Those skilled in the art will recognize there are several second vascular intervention devices known in the art that are within the spirit and scope of the present invention.

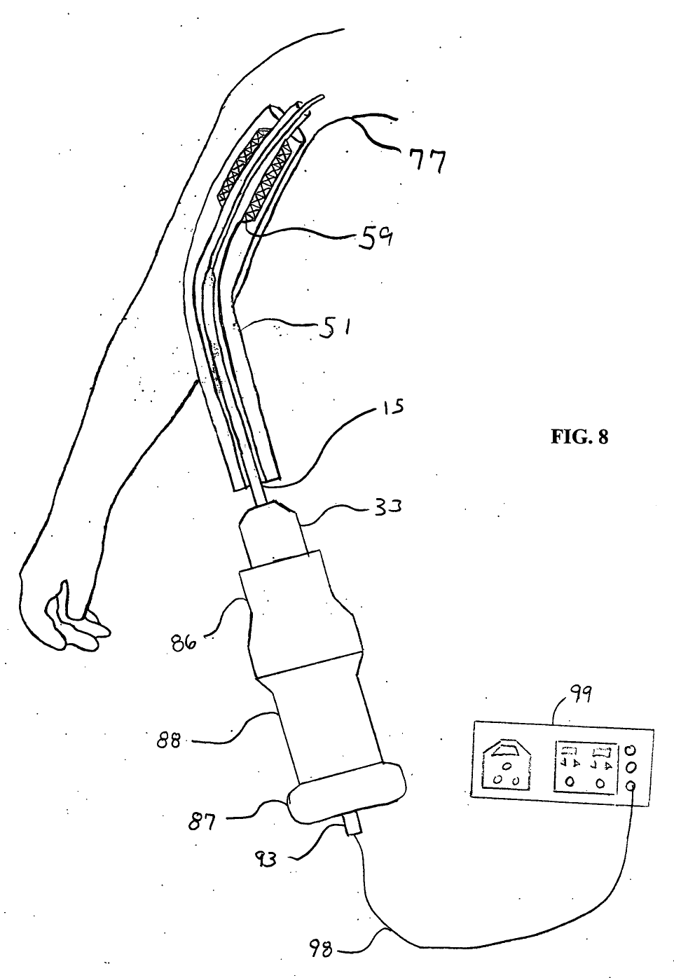

FIG. 8 shows the ultrasonic probe 15 with the first vascular intervention device 51 and the second vascular intervention device 59 placed over a portion of the longitudinal axis of the ultrasonic probe 15 proximal to a site of the occlusion. The second vascular intervention device 59 is moved inside of the first vascular intervention device 51 and moved proximal to the site of the occlusion.

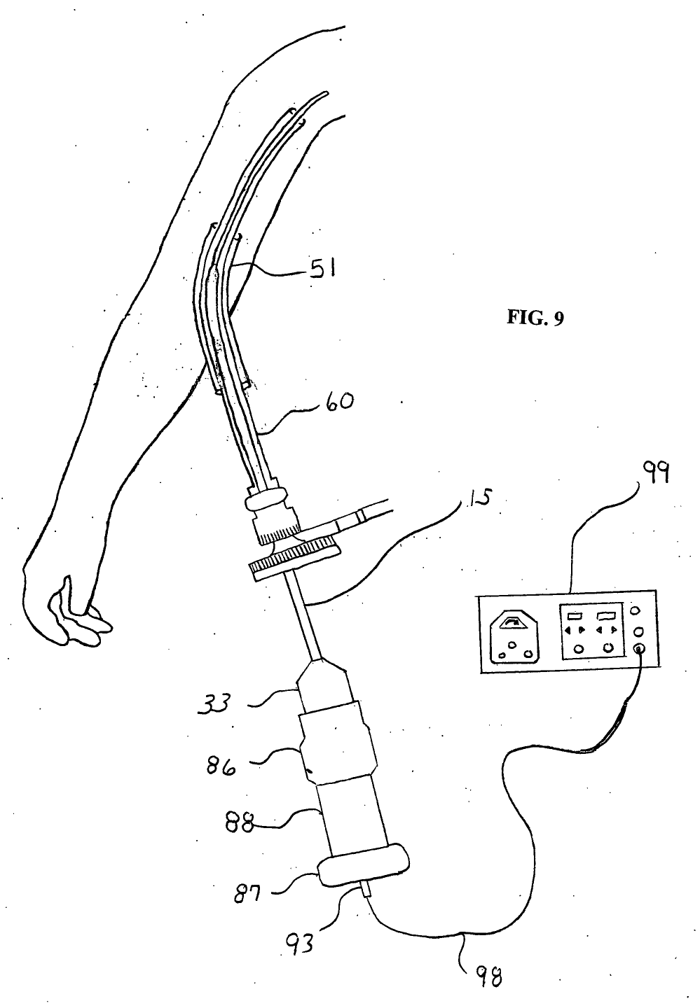

FIG. 9 shows the ultrasonic probe 15 with the first vascular intervention device 51 and an alternative second vascular intervention device 60 placed over a portion of the longitudinal axis of the ultrasonic probe 15 proximal to the site of the occlusion. In the embodiment of the present invention shown in FIG. 9, the second vascular intervention device 60 is used to deliver a pharmacological agent to the site of the occlusion.

FIGS. 8-9 show a final stage of inserting the ultrasonic probe 15, the first vascular intervention device 51 and a second vascular intervention device 59, 60 to the site of the occlusion. There are several ways to deliver the ultrasonic probe 15, the first vascular intervention device 51 and the second vascular intervention device 59 to the site of the occlusion.

In one embodiment of the present invention, the ultrasonic probe 15 is inserted into the vasculature in the arm 77 by grasping the handle 88 and inserting the ultrasonic probe 15 into the vasculature and moving the ultrasonic probe 15 proximal to the site of the occlusion. The ultrasonic probe 15 is uncoupled from the transducer 22 by unfastening the quick attachment-detachment system 33 from the transducer 22, exposing the proximal end 31 of the ultrasonic probe. The first vascular intervention device 51 is placed over the small proximal end 31 of the ultrasonic probe 15 and moved over a portion of the longitudinal axis of the ultrasonic probe 15. The second vascular intervention device is then placed over the proximal end 31 of the ultrasonic probe 15 and moved over the longitudinal axis of the ultrasonic probe within the first vascular intervention device.

In another embodiment of the present invention, a guidewire is inserted into the vasculature of the body and a first vascular intervention device 51 is placed over a longitudinal axis of the guidewire. After the guidewire is removed from the vasculature, the ultrasonic probe 15 is inserted into the first vascular intervention device 51 by grasping the handle 88 and inserting the ultrasonic probe 15 into the vasculature and moving the ultrasonic probe 15 to the treatment site. The proximal end 31 of the ultrasonic probe 15 is exposed by disengaging the ultrasonic probe 15 from the transducer 22 while the ultrasonic probe 15 remains in the vasculature at the site of the occlusion. A second vascular intervention device 59, 60 is placed over the proximal end 31 of the ultrasonic probe 15 and moved within the interior of the first vascular intervention device 51 over the longitudinal axis of the ultrasonic probe 15. The ultrasonic probe 15 is engaged to the transducer 22 with the quick attachment-detachment system 33 and the ultrasonic energy source 99 is activated to ablate the occlusion in the arm 77.

The present invention provides a method of placing a first vascular intervention device 51 over an ultrasonic probe 15 and moving the first vascular intervention device 51 to a treatment site. The ultrasonic probe 15 is inserted into the vasculature of the body to the treatment site and a quick attachment-detachment system that is coupled to the proximal end 31 of the ultrasonic probe 15 is uncoupled. A first vascular intervention device 51 is placed over a small diameter at the proximal end 31 of the ultrasonic probe 15 and moved along the longitudinal axis of the ultrasonic probe until the first vascular intervention device 51 is adjacent to the treatment site while the ultrasonic probe 15 remains in an approximately fixed position in the vasculature.

The ultrasonic probe 15 is placed in communication with the biological material by moving, sweeping, bending, twisting or rotating the ultrasonic probe 15 along the biological material. Those skilled in the art will recognize that the many ways to move the ultrasonic probe in communication with the biological material known in the art are within the spirit and scope of the present invention.

The present invention provides a method of exchanging vascular intervention devices within a vasculature of the body in a time efficient manner. A first vascular intervention device 51 is inserted into the vasculature and an ultrasonic probe 15 is delivered within the first vascular intervention device 51 to the treatment site. A second vascular intervention device 59, 60 is moved over the proximal end 31 of the ultrasonic probe 15 while the ultrasonic probe 15 remains in an approximately fixed position in the vasculature. The second vascular intervention device 59, 60 is moved within an interior of the first vascular intervention device 51 and along the longitudinal axis of the ultrasonic probe 15 to the treatment site.

In an alternative embodiment of the present invention, the ultrasonic probe 15 is vibrated in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985, and the entirety of this application is hereby incorporated herein by reference.

In another embodiment of the present invention, the ultrasonic probe 15 is vibrated in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active area of the ultrasonic probe 15, creating a plurality of nodes and a plurality of anti-nodes along the active area that result in cavitation in a medium surrounding the ultrasonic probe 15. The active area of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active area of the ultrasonic probe 15 is vibrated in a direction not parallel to the longitudinal axis of the ultrasonic probe 15 while equally spaced points along the longitudinal axis of the ultrasonic probe 15 vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898, and the entirety of this application is hereby incorporated herein by reference.

The present invention provides an apparatus and a method for an ultrasonic medical device 11 having a probe 15 with a small proximal end 31. The present invention allows for a vascular intervention device to be moved over the ultrasonic probe 15 while the ultrasonic probe remains in an approximately fixed position in the vasculature. The present invention provides an apparatus and a method for an ultrasonic medical device 11 having a probe 15 with a small proximal end 31 that is simple, reliable, user friendly and allows for the exchange of vascular intervention devices in a time efficient manner.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device comprising:
an ultrasonic probe having a proximal end, a distal end, and a longitudinal axis therebetween;
a transducer having a proximal end and a distal end, the transducer transmitting an ultrasonic energy to the ultrasonic probe; and
a coupling that engages the proximal end of the ultrasonic probe to the distal end of the transducer; and
an ultrasonic energy source engaged to the transducer that produces the ultrasonic energy,
wherein the proximal end of the ultrasonic probe has a small diameter that allows a vascular intervention device to be placed over the proximal end of the ultrasonic probe and moved along the longitudinal axis of the ultrasonic probe while the ultrasonic probe remains within a vasculature of a body.

2. The ultrasonic medical device of claim 1 wherein a diameter of the ultrasonic probe varies from the proximal end of the ultrasonic probe to the distal end of the ultrasonic probe.

3. The ultrasonic medical device of claim 1 wherein a diameter of the ultrasonic probe is approximately uniform from the proximal end of the ultrasonic probe to the distal end of the ultrasonic probe.

4. The ultrasonic medical device of claim 1 further comprising at least one transition along the longitudinal axis of the ultrasonic probe to change a diameter from the proximal end to the distal end.

5. The ultrasonic medical device of claim 4 wherein at least one transition gradually changes the diameter from the proximal end to the distal end along the longitudinal axis of the ultrasonic probe.

6. The ultrasonic medical device of claim 4 wherein at least one transition is stepwise to change the diameter from the proximal end to the distal end along the longitudinal axis of the ultrasonic probe.

7. The ultrasonic medical device of claim 1 wherein a diameter of the ultrasonic probe slowly tapers from the proximal end to the distal end along the longitudinal axis of the ultrasonic probe.

8. The ultrasonic medical device of claim 1 wherein the coupling further comprises a base and a housing that engages the base.

9. The ultrasonic medical device of claim 1 wherein the coupling disengages the proximal end of the ultrasonic probe from the transducer to allow the vascular intervention device to be placed over the ultrasonic probe.

10. The ultrasonic medical device of claim 1 wherein the ultrasonic probe remains in an approximately fixed position in the vasculature when the vascular intervention device is placed over the proximal end of the ultrasonic probe.

11. The ultrasonic medical device of claim 1 wherein the small diameter at the proximal end of the ultrasonic probe is approximately uniform along a length of the proximal end of the ultrasonic probe.

12. The ultrasonic medical device of claim 1 wherein the vascular intervention device is selected from a group consisting of a balloon catheter, a PTCA balloon, a stent, a stent delivery system, a graft, a stent graft, a drug eluding stent and a catheter.

13. The ultrasonic medical device of claim 1 wherein the small diameter at the proximal end of the ultrasonic probe is less than approximately 0.035 inches.

14. The ultrasonic medical device of claim 1 wherein the ultrasonic probe is disposable.

15. The ultrasonic medical device of claim 1 wherein the ultrasonic probe is for a single use on a single patient.

16. The ultrasonic medical device of claim 1 wherein a transverse ultrasonic vibration generates a plurality of transverse nodes and a plurality of transverse anti-nodes along at least a portion of the longitudinal axis of the ultrasonic probe.

17. The ultrasonic medical device of claim 1 wherein the ultrasonic probe comprises a material that allows the ultrasonic probe to be bent, flexed and deflected.

18. The ultrasonic medical device of claim 1 wherein the ultrasonic energy source delivers ultrasonic energy in a frequency range from about 10 kHz to about 100 kHz.

19. An elongated ultrasonic probe comprising:
a proximal end, a distal end terminating in a probe tip and a longitudinal axis between the proximal end and the distal end; and
a small diameter at the proximal end;
wherein the small diameter allows for a first vascular intervention device to be placed over the proximal end of the elongated ultrasonic probe and moved along the longitudinal axis of the elongated ultrasonic probe while the distal end of the elongated ultrasonic probe remains in a vasculature of a body.

20. The device of claim 19 wherein the first vascular intervention device is selected from a group consisting of a balloon catheter, a PTCA balloon, a stent, a stent delivery system, a graft, a stent graft, a drug eluding stent and a catheter.

21. The device of claim 19 wherein the elongated ultrasonic probe is a guide for the first vascular intervention device and a second vascular intervention device.

22. The device of claim 19 wherein the small diameter of the elongated ultrasonic probe allows for a second vascular intervention device to be moved inside the first vascular intervention device.

23. The device of claim 19 wherein the second vascular intervention device is selected from a group consisting of a balloon catheter, a PTCA balloon, a stent, a drug eluding stent, a catheter, a probe and a lumen.

24. The device of claim 19 wherein a diameter of the elongated ultrasonic probe is approximately uniform from the proximal end of the elongated ultrasonic probe to the distal end of the elongated ultrasonic probe.

25. The device of claim 19 wherein a diameter of the elongated ultrasonic probe varies from the proximal end of the elongated ultrasonic probe to the distal end of the elongated ultrasonic probe.

26. The device of claim 19 further comprising at least one transition along the longitudinal axis of the elongated ultrasonic probe to change a diameter from the proximal end to the distal end.

27. The device of claim 19 wherein the elongated ultrasonic probe is for a single use on a single patient.

28. The device of claim 19 wherein the elongated ultrasonic probe is disposable.

29. A method of placing a first vascular intervention device over an ultrasonic probe comprising:
inserting an ultrasonic probe into a vasculature of a body;
moving the ultrasonic probe to the treatment site;
disengaging a coupling that engages a proximal end of the ultrasonic probe and a transducer to expose the proximal end of the ultrasonic probe;
placing the first vascular intervention device over a small diameter at the proximal end of the ultrasonic probe;
moving the first vascular intervention device along a longitudinal axis of the ultrasonic probe so the first vascular intervention device is adjacent to the treatment site while the ultrasonic probe remains in an approximately fixed position in the vasculature;
re-engaging the coupling to engage the proximal end of the ultrasonic probe and the transducer;
activating an ultrasonic energy source to provide an ultrasonic energy to the ultrasonic probe; and
ablating an occlusion at the treatment site with the ultrasonic probe.

30. The method of claim 29 wherein the first vascular intervention device is selected from a group consisting of a balloon catheter, a PTCA balloon, a stent, a stent delivery system, a graft, a stent graft, a drug eluding stent and a catheter.

31. The method of claim 29 wherein the small diameter at the proximal end of the ultrasonic probe is located outside of the vasculature when the coupling is disengaged from the ultrasonic probe.

32. The method of claim 29 further comprising exposing the small diameter of the ultrasonic probe when disengaging the coupling from the proximal end of the ultrasonic probe.

33. The method of claim 29 wherein a diameter at the proximal end of the ultrasonic probe is approximately uniform along a length of the proximal end of the ultrasonic probe.

34. The method of claim 29 wherein the ultrasonic probe guides for the first vascular intervention device to the occlusion.

35. The method of claim 29 wherein a diameter of the ultrasonic probe is approximately uniform from the proximal end of the ultrasonic probe to a distal end of the ultrasonic probe.

36. The method of claim 29 wherein a diameter of the ultrasonic probe varies form the proximal end of the ultrasonic probe to a distal end of the ultrasonic probe.

37. The method of claim 29 wherein the ultrasonic probe comprises at least one transition along the longitudinal axis of the ultrasonic probe to change a diameter from the proximal end to a distal end of the ultrasonic probe.

38. The method of claim 29 further comprising moving a second vascular intervention device inside the first vascular intervention device.

39. The method of claim 38 further comprising removing the first vascular intervention device from the vasculature.

40. A method of exchanging vascular intervention devices within a vasculature of a body comprising:
inserting a first vascular intervention device into the vasculature;
delivering a flexible ultrasonic probe inside of the first vascular intervention device to a treatment site;
moving a second vascular intervention device over a proximal end of the flexible ultrasonic probe while the flexible ultrasonic probe remains in an approximately fixed position in the vasculature; and
moving the second vascular intervention device within an interior of the first vascular intervention device along a longitudinal axis of the flexible ultrasonic probe to the treatment site.

41. The method of claim 40 wherein the proximal end of the flexible ultrasonic probe has a small diameter.

42. The method of claim 40 wherein the proximal end of the flexible ultrasonic probe is outside of the vasculature when the second vascular intervention device is placed over the proximal end of the flexible ultrasonic probe.

43. The method of claim 40 wherein the small diameter at the proximal end of the flexible ultrasonic probe is located outside of the body when the second vascular intervention device is placed within the first vascular intervention device.

44. The method of claim 40 wherein the flexible ultrasonic probe is a guide for the second vascular intervention device.

45. The method of claim 40 wherein a diameter of the flexible ultrasonic probe is approximately uniform from the proximal end of the flexible ultrasonic probe to the distal end of the flexible ultrasonic probe.

46. The method of claim 40 wherein a diameter of the flexible ultrasonic probe varies from the proximal end of the flexible ultrasonic probe to the distal end of the flexible ultrasonic probe.

47. The method of claim 40 wherein the flexible ultrasonic probe is for a single use on a single patient.

48. The method of claim 40 wherein the flexible ultrasonic probe is disposable.
